(19) United States
(12) Patent Application Publication
(10) Pub. No.: US 2007/0270717 A1
(43) Pub. Date: Nov. 22, 2007

(54) MULTI-FACETED OPTICAL REFLECTOR

(75) Inventors: Jing Tang, Arlington, MA (US); Timothy M. Looney, Tyngsboro, MA (US); S. Eric Ryan, Hopkinton, MA (US)

Correspondence Address: MILLS & ONELLO LLP, ELEVEN BEACON STREET, SUITE 605, BOSTON, MA, 02108, US

(73) Assignee: CORNOVA, INC., Burlington, MA (US)

(21) Appl. No.: 11/834,096

(22) Filed: Aug. 6, 2007

Related U.S. Application Data
(63) Continuation-in-part of application No. 11/537,258, filed on Sep. 29, 2006.
(60) Provisional application No. 60/722,753, filed on Sep. 30, 2005.
(60) Provisional application No. 60/761,649, filed on Jan. 24, 2006.
(60) Provisional application No. 60/823,812, filed on Aug. 29, 2006.
(60) Provisional application No. 60/824,915, filed on Sep. 8, 2006.
(60) Provisional application No. 60/823,812, filed on Aug. 29, 2006.
(60) Provisional application No. 60/824,915, filed on Sep. 8, 2006.
(60) Provisional application No. 60/821,623, filed on Aug. 7, 2006.
(60) Provisional application No. 60/884,630, filed on Jan. 12, 2007.

(51) Int. Cl. A61B 5/00; C03B 37/023; G02B 6/26

(52) U.S. Cl. 600/585; 385/31; 65/385

(57) ABSTRACT

A reflecting element with multiple reflective facets is integrated with the distal end of a multi-fiber optical probe. The facets are shaped depending on the type of analysis performed and according to the desired distribution of radiation to and from internal body tissues and fluids. The probe can include a protective transparent balloon or other covering that separates the reflecting element from interior tissue walls and provides a window for radiation to be transmitted between the reflecting facets and a region of interest. The probe can be integrated with treatment-based devices, including lumen-expanding angioplasty balloon catheters. The probe can also be adapted as an imaging device such as an endoscope.

### RELATED APPLICATIONS

This application is a continuation-in-part application of U.S. Ser. No. 11/537,258, filed on Sep. 29, 2006, and published as U.S. Patent Publication Number 2007/0078500 A1, which claims the benefit of U.S. Provisional Patent Application No. 60/722,753 filed on Sep. 30, 2005, U.S. Provisional Patent Application No. 60/761,649 filed on Jan. 24, 2006, U.S. Provisional Patent Application No. 60/823,812 filed on Aug. 29, 2006, and U.S. Provisional Patent Application No. 60/824,915 filed on Sep. 8, 2006, the contents of each of which is incorporated herein by reference in its entirety.

This application also claims the benefit of U.S. Provisional Patent Application No. 60/821,623 filed on Aug. 7, 2006 and U.S. Provisional Patent Application No. 60/884,630 filed Jan. 12, 2007, the contents of each of which is incorporated herein by reference in its entirety.

### FIELD OF THE INVENTION

The invention relates to optical components for fiber optic probes including endoscopic and/or catheterized devices for optically analyzing or treating internal body lumens and tissues.

### BACKGROUND OF THE INVENTION

Many serious health conditions can be attributed to diseased or damaged conditions of interior vessels. For example, vascular lesions, aneurysms, and the build-up of plaque within interior vessel walls may rupture or cause blockages that result in heart attacks. A number of treatments (e.g. angioplasty) for these conditions have been developed, but they critically rely on first detecting the debilitating conditions. Developing methods for detecting these conditions and also for treating them often rely on high-precision, low-profile optical systems suitable for insertion into small body lumens and cavities.

Traditional techniques for analyzing lumen walls include x-ray fluoroscopy and intravascular ultrasound. These techniques, however, are expensive and/or may cause harmful side-effects. Less harmful technologies for characterizing tissue in body vessels have been developed, such as catheters or endoscopes with integrated fiber-optics. Such fiber optic probes are generally designed with one or more fibers extending to the distal end of the catheter, some designated for transmitting radiation and some for collecting radiation (see, e.g., U.S. Pat. No. 5,106,387 by Kittrell. Light collected from the probe may be analyzed in various ways, including by direct observance or with more elaborate devices such as spectrometers. Many adaptations of these fiber-optic arrangements, however, include drawbacks in their design which diminish their ability to reliably assess certain types of tissue conditions.

For example, erosion in the cells of a lumen, grown over by other tissue material, may be difficult to detect from radiation emitted directly back from the lumen wall along substantially the same optical path that the radiation was first transmitted. It is therefore desirable in certain cases to accurately control the direction of radiation such that it is incident on tissue at predetermined offset angles and collected at other predetermined angles relative to the tissue. In addition, where delivery and collection fibers are spaced closely together, radiation from the source fiber may leak to the collection fiber, creating noise or otherwise negatively affecting the results. It is therefore advantageous for the ends of delivery and collection fibers to be separated.

U.S. Pat. No. 6,485,413 by Boppart, et al. shows a number of designs for directing radiation through predetermined paths between transmission and collection conduits employing various optical elements (e.g. gradient index (GRIN) lenses, multiple rotating prisms, etc.), including those having actuating mechanical arrangements (e.g. movable cantilevered arms or pneumatic devices). Accurate relative placement or design of these optics potentially adds significant expense and/or time to the assembly and manufacturing process. Another type of arrangement, as shown in U.S. Pat. No. 6,701,181 by Tang, et al., teaches the use of multiple separate cone-shaped optical redirectors, each limited to directing light in a particular manner to separated conical pieces. The region illuminated by such an assembly may unpredictably and undesirably change longitudinally with respect to a catheter if the location of the region changes radially (outwardly). Another system characterized in U.S. Pat. No. 6,873,868 by Furnish shows an elongated assembly including at least 8-12 annularly-disposed open grooves for placement and alignment of corresponding delivery and/or collection fibers. Furnish also includes redirecting components that are at the ends of grooves, and have shapes (i.e. width, depth) substantially conforming to that of the grooves, thus limiting the redirecting scope of the redirecting component for each fiber. Limited redirecting scope associated with each fiber can necessitate the use of a relatively high number of fibers (8-12) in order to provide broad coverage. Subsequently, the diameter, length, and stiffness of the optical assembly due to the high number of fibers can have undesirable effects, such as prohibitively limiting the devices' flexibility and ability to enter narrow (e.g., less than about 1.5 mm) curvilinear passageways, especially in relation to intravascular applications, including coronary applications. Manufacture of the faceted element using conventional methods, such as those disclosed in Furnish, can be complicated and expensive since the alignment grooves and the facets are formed from a single piece of material.

Other devices also provide for controlled delivery of radiation for other purposes, including treatment, and similarly require high-precision, low-profile optics. For example, U.S. Pat. No. 5,304,173 by Kittrell, et al. characterizes a catheter system combining intravascular spectral diagnostics and laser treatment of atherosclerotic disease. U.S. Pat. No. 5,997,570 by Ligtenberg et al. provides a method for introducing a stent with a fiber optics-integrated balloon catheter system for deploying and curing the stent in-vivo with radiation. All publications, patent applications, patents, and other references mentioned herein are incorporated by reference in their entirety. In case of conflict, the present specification, including definitions, will control.

Many endoscopic devices are also encumbered because of optics that provide limited viewing perspectives and/or require manual manipulation in order to provide more complete views of surrounding tissues or lumens. The imaging end of endoscopes generally include an optical cylinder attached to a viewing system (e.g. similar to a camera) with a limited angular scope that must be rotated or otherwise manipulated in order to provide more complete pictures.

It is therefore desirable to have more simplified and cost-effective methods and designs for controlling the direction of transmission and collection of radiation in catheter/endoscope-based optical analysis and viewing systems.

### SUMMARY OF THE INVENTION

The systems and methods of the invention provide hospitals and physicians with reliable, simplified, and cost-effective optical components for body lumen inspection devices, including catheter and endoscopic-based devices useful for diagnosing a broad range of tissue conditions. Embodiments of the invention provide reliable control over multiple light emission paths within a multiple-fiber catheter and/or endoscopic probe while allowing the probe to remain substantially flexible and maneuverable within a body lumen. Reliance on inflexible, expensive, elaborate and/or difficult to assemble components that inhibit prior art devices is thus reduced. By improving control over light emission paths and reducing inadvertent signal leaking or losses with efficient and low profile components, fewer fibers are required than with typical prior art devices. Thus, improving the flexibility and reducing the size of such a system is especially beneficial for small body vessel applications.

In an aspect of the invention, an endovascular probe assembly for guiding light radiation therein comprises at least one waveguide, a conduit having a longitudinal axis, wherein the at least one waveguide is positioned on a surface of the conduit, and extends along the longitudinal axis of the conduit, and a reflecting element about the conduit. The reflecting element has a plurality of reflective facets formed out of an end of the reflecting element and that are annularly arranged about the conduit. Each of the reflective facets is positioned to at least one of direct light to the at least one waveguide and receive reflected light from the at least one waveguide.

In an embodiment, the reflecting element is substantially cylindrically shaped.

In an embodiment, the at least one waveguide consists of 4 or fewer waveguides. In another embodiment, the 4 or fewer waveguides consists of 4 waveguides.

In an embodiment, the reflective facets are planar and have predetermined angles with respect to the longitudinal axis of the conduit. In an embodiment, at least one of the predetermined angles of one of the reflective facets is distinct from a predetermined angle of at least one other reflective facet of the plurality of reflective facets.

In an embodiment, the reflective facets are shaped according to one or more predetermined polynomials.

In an embodiment, the reflecting element is metallic.

In an embodiment, the reflecting element is comprised of a polymer and the face of each reflective facet includes a highly reflective surface layer. In an embodiment, the highly reflective surface layer is selected from the group consisting of steel, nickel, aluminum, titanium, platinum, gold, silver, and alloys therefrom.

In an embodiment, the reflecting element has a maximum longitudinal length of about half a millimeter or less.

In an embodiment, the reflecting element includes an opening having an inner surface, wherein the conduit is inserted through the opening, and further includes an outer surface, wherein a surface of each facet extends from the inner surface to the outer surface.

In an embodiment, the reflecting element has a maximum outer diameter of about a millimeter or less.

In an embodiment, the reflective element is contained within a partial covering of the distal end, partial covering being substantially transparent to a predetermined range of radiation. In an embodiment, the partial covering is a flexible angioplasty-type balloon.

In an embodiment, the probe assembly further comprises an alignment segment for aligning the distal ends of the at least one waveguide with the reflective facets. In an embodiment, the alignment segment includes one or more holes through which the ends of the at least one waveguide can be passed and aligned with the reflective facets. In an embodiment, the alignment segment includes one or more grooves for aligning the ends of the at least one waveguide with the reflective facets. In an embodiment, the alignment segment has a longitudinal length of about 350 micrometers or less.

In an embodiment, the probe assembly comprises columns between the reflective facets that substantially block radiation from traveling directly between waveguides corresponding to distinct reflective facets.

In an embodiment, an intervening cavity is positioned between adjacent reflective facets.

In an embodiment, at least one individual waveguide is arranged to at least one of deliver light to multiple reflective facets and collect light from multiple reflective facets.

In an embodiment, at least one facet is of a different size than at least one other facet of the plurality of facets. In an embodiment, the at least one facet of a different size is of a larger size than the at least one other facet and is arranged to direct light to a collection fiber.

In an embodiment, the at least one waveguide is at least one optical fiber.

In an embodiment, the at least one waveguide arrangement and the at least one reflecting element are configured for collecting images. In an embodiment, the fiber optic probe assembly is adapted for use in an endoscope.

In an aspect of the invention, a method of inspecting a body lumen is provided. The method includes providing a source of radiation to a catheter probe having a reflecting element at its distal end, the reflecting element having a plurality of reflective facets that are annularly arranged about the reflecting element and that are formed out of an end of the reflecting element, wherein each reflective facet is shaped to at least one of deliver radiation and receive radiation via one or more corresponding waveguides to or from a target area at predetermined angles of incidence. The method further includes inserting the catheter probe into a target area, collecting through the catheter probe radiation that is received by the reflective facets from the target area, and delivering the received radiation to an analyzer or imager.

In an embodiment, the target area is a body lumen. In an embodiment, the body lumen has a diameter of about 4 millimeters or less.

In an embodiment, inserting the catheter probe includes inserting an angioplasty-balloon integrated with the catheter probe, wherein the balloon seals the reflecting element from body tissue and fluid, and is substantially transparent to radiation from the catheter's radiation source. In an embodiment, inserting the catheter probe includes prior to receiving the radiation, filling the balloon with a non-toxic liquid, and expanding the balloon, wherein a substantial portion of the external surface of the balloon is pressed against body-lumen tissue.

In an aspect of the invention, a method for making a fiber-optic probe assembly is provided. The method includes forming a conduit having a longitudinal axis, wherein a waveguide arrangement is positioned on a surface of the conduit, and extends along the longitudinal axis of the conduit. The method further includes forming a reflecting element about the conduit, the reflecting element comprising a plurality of reflective facets, wherein the reflective facets are formed out of an end of the reflecting element and arranged annularly about the reflecting element, and each reflective facet is positioned to at least one of direct radiation to the waveguide arrangement and receive light from the waveguide arrangement.

In an embodiment, the waveguide arrangement includes at least one delivery optical fiber and at least one collection optical fiber.

In an embodiment, the method for making a fiber-optic probe assembly further includes forming a protective covering about the reflecting element, the covering being substantially transparent to at least a predetermined range of radiation.

In an embodiment, forming the reflecting element includes forming a cylindrical ring out of metal and forming flat reflective facets out of an end of the cylindrical ring. In an embodiment, the flat reflective facets are formed out of the cylindrical ring with a polisher.

In an embodiment, the reflecting element is formed out of a pre-fabricated mold. In an embodiment, the reflecting element is molded out of plastic and the facets of the plastic, molded reflecting element are subsequently layered with a thin reflecting coating.

In an embodiment, the non-facet surfaces of the reflecting element are coated with an anti-reflecting coating.

In an aspect of the invention, an endoscope assembly for guiding optical radiation therein is provided that comprises a conduit along which an waveguide arrangement extends, and includes one or more reflecting elements that are disposed about the conduit, each of the reflecting elements having a plurality of reflective facets arranged and configured to at least one of direct light to and receive light from the waveguide arrangement.

In an embodiment, the waveguide arrangement and at least one of the reflecting elements are arranged and configured to collect images.

In an aspect of the invention, a probe assembly for guiding light radiation therein comprises at least one waveguide, a conduit having a longitudinal axis, wherein the at least one waveguide is positioned on the conduit, and extends along the longitudinal axis of the conduit, and a reflecting element about the conduit. The reflecting element comprises a opening having an inner surface, wherein the conduit is inserted through the opening. The reflecting element has an outer surface, and the reflecting element comprises a plurality of reflective facets, wherein a surface of each facet extends from the inner surface to the outer surface, and wherein each of the reflective facets is positioned to at least one of direct light to the at least one waveguide and receive reflected light from the at least one waveguide.

In an embodiment, at least one individual waveguide is arranged to at least one of deliver light to multiple reflective facets and collect light from multiple reflective facets.

In an embodiment, the reflecting element has a maximum longitudinal length of about half a millimeter or less.

In an embodiment, the reflecting element has a maximum diameter of about a millimeter or less.

In an embodiment, the reflecting element comprises a maximum of six facets.

In an embodiment, t the reflecting element comprises a maximum of four facets.

In an embodiment, the outer diameter of the reflecting element is about a millimeter or less.

In an aspect of the invention, a probe conduit is provided having a reflecting element with a plurality of facets formed out of an end of the element. The facets are substantially aligned with one or more waveguides. In an embodiment, the reflecting element is substantially cylindrically shaped and encircles an end of the conduit along which the optical fibers extend. The cylindrical shape is especially suited for integration with similarly cylindrical catheter probes and for distribution and collection of radiation about a circumference of the reflecting element and a catheter probe in which it can be integrated. The facets are formed out of the reflecting element and may be arranged to face the ends of corresponding optical fibers, directing light to and from the fibers in predetermined directions. In another embodiment of the invention, the reflecting element is integrated with an alignment segment for aligning waveguides with the facets. In an embodiment of the invention, the alignment segment includes holes through which waveguides are held and aligned with the facets. In another embodiment, the alignment segment includes open grooves which can hold and align the waveguides with respect to the facets. In another embodiment, separators are positioned between the facets so that undesired light transmissions traveling directly between the facets and/or waveguides are substantially reduced.

In embodiments of the invention, the facets can be of various shapes and configurations. In an embodiment of the invention, facets are substantially planar at predetermined angles with respect to the conduit. In another embodiment of the invention, the facets are curvilinear-shaped according to one or more predetermined polynomials. In other embodiments of the invention, multiple facets share a single fiber. A single fiber can deliver and/or collect radiation across multiple facets within, for example, a 100 degree circumferential span. In yet another embodiment of the invention, multiple fibers share a single facet.

In embodiments of the invention, the reflecting element can be formed and/or coated with various materials. In an embodiment of the invention, the reflecting element is metallic. The facets can be shaped out of the reflecting element and then finished and/or polished according to need. In another embodiment of the invention, the reflecting element is formed out of plastic or similar material. The facets can then be coated with reflective material such as, for example, metallic materials including steel, nickel, aluminum, gold, and alloys therefrom. Aspects of the reflecting element not intended for reflecting radiation can be coated with an anti-reflective material so as to minimize glare and noise.

In an embodiment of the invention, the dimensions of the reflecting element are optimized for allowing maximum flexibility of the conduit and the ability to pass through narrow lumens (e.g., coronary vessels). In an embodiment of the invention, the reflecting element has a maximum diameter of about a millimeter or less. In another embodiment of the invention, the reflecting element has a longitudinal length of about half a millimeter or less.

In another aspect of the invention, a probe conduit has at its distal end a reflecting element with multiple reflective facets for directing light to or from less than 8 waveguides. In an embodiment of the invention, 2 delivery and 2 collection waveguides can provide data collection for at least 4 separate regions about the circumference of the conduit.

In embodiments of the invention, the probe conduit includes a protective outer covering allowing radiation to pass between the reflective facets and targeted areas about the outside of the covering such as, for example, vessel walls. In an embodiment of the invention, the protective outer covering comprises a flexible expandable balloon such as, for example, an angioplasty-type balloon. In another embodiment of the invention, the covering comprises a solid transparent covering formed out of, for example, plastic.

In another aspect of the invention, a method is provided for inspecting a body lumen which includes providing a source of radiation to a catheter probe having an embodiment of the reflecting element such as, for example, the apparatus embodiments described above, positioning the catheter probe within the lumen area targeted for inspection, delivering radiation to the area targeted for inspection via the reflecting element, collecting radiation from the targeted area via the reflecting element, and directing the collected radiation to an analyzer/imager.

In an embodiment, wherein the catheter probe includes an angioplasty-type balloon such as referred to above, is the method further includes filling and expanding the angioplasty-type balloon with a substantially clear non-toxic fluid so as to press the outside surface of the balloon against the walls of the target area while permitting radiation to travel between the conduit and walls of the target area. Fluids may include, for example, saline solution.

In another aspect of the invention, a method is provided for making a fiber-optic probe assembly comprising forming a reflective element for placement about a conduit that has a waveguide arrangement thereon, wherein the reflective element has a plurality of facets about an end of the element, and positioning the reflective element onto the conduit wherein at least one facet is positioned to be substantially aligned with a delivery waveguide or collection waveguide.

In an embodiment, the above method includes aligning the waveguide arrangement with the plurality of facets comprising: integrating the ends of waveguides from the waveguide arrangement with an alignment segment; and fixedly aligning the alignment segment with the plurality of facets. In an embodiment of the invention, the alignment segment comprises an open grooved segment. In another embodiment, the alignment segment comprises a holed segment having a plurality of holes through which the ends of the waveguides are passed through. In another embodiment of the invention, the alignment segment is first aligned with the facets prior to integration with the waveguides.

In embodiments, forming the reflective element includes forming facets out of a cylindrical ring with, for example, a polisher. In another embodiment, forming the reflective element includes forming a pre-fabricated mold. In a further embodiment, the facets are layered with one or more thin reflective coatings. In an embodiment, forming the reflective element includes first coating the non-reflective surfaces of the reflecting element with an anti-reflective coating.

In another aspect of the invention, an endoscope assembly is provided including a conduit along which a waveguide arrangement extends. One or more reflecting elements disposed about the conduit, each of the reflecting elements having a plurality of reflective facets for at least one of directing light to and from the waveguide arrangement. In an embodiment, at least one of the reflecting elements is arranged and configured for collecting endoscopic images.

### BRIEF DESCRIPTION OF THE DRAWINGS

The structure, operation, and methodology of the invention, together with other objects and advantages thereof, may best be understood by reading the following detailed description in connection with the drawings in which each part has an assigned numeral or label that identifies it wherever it appears in the various drawings. The drawings are not necessarily to scale, emphasis instead being placed upon illustrating the principles of the invention.

FIG. 1 is an illustrative view of a balloon catheter assembly with integrated optics incorporating an embodiment of the present invention.

FIG. 2A is an illustrative elevated perspective view of a reflecting element in accordance with the invention.

FIG. 2B is an illustrative transverse view of the reflecting element shown in FIG. 2A.

FIG. 2C is an illustrative side perspective view of the embodiment of FIG. 2A.

FIG. 3A is an illustrative view of the distal end of a balloon catheter probe with integrated optics in accordance with an embodiment of the invention.

FIG. 3B is an expanded illustrative view of the invention as shown in FIG. 3A.

FIG. 4 is an illustrative view of a catheter probe with integrated optics having a solid transparent window in accordance with an embodiment of the invention.

FIG. 5A is an illustrative view of an embodiment of the invention having a 4-facet reflecting element integrated into the distal portion of a balloon catheter in accordance with an embodiment of the invention.

FIG. 5B is a cross-sectional illustrative view of the balloon catheter shown in FIG. 4A in accordance with an embodiment of the invention.

FIG. 6A is an illustrative view of the distal end of a balloon catheter with an alternate catheter packaging arrangement in accordance with an embodiment of the invention.

FIG. 6B is a cross-sectional illustrative view of the balloon catheter shown in FIG. 6A in accordance with an embodiment of the invention.

FIG. 7 is an illustrative view of the catheter of FIG. 6A showing sample trace lines of emission and collection rays in accordance with an embodiment of the invention.

FIGS. 8A-8D are illustrative views of sample emission/collection trace lines between a fiber and facet according to an embodiment of the invention.

FIGS. 9A-9B are illustrative elevated perspective and transverse views, respectively, of a 6-facet reflecting element according to an embodiment of the invention.

FIGS. 10A-B are illustrative elevated perspective views of an embodiment of the invention having light-blocking separators positioned between facets.

FIG. 11A is an illustrative elevated perspective view of a fiber alignment ring having fiber alignment holes according to an embodiment of the invention.

FIG. 11B is an illustrative side perspective view of the embodiment of FIG. 11A.

FIG. 11C is an illustrative transverse view of the embodiment of FIG. 11A.

FIG. 11D-11E are illustrative elevated perspective and transverse views, respectively, of an embodiment of the invention with a distinct alignment segment including fiber retaining grooves.

FIG. 12A is an illustrative side perspective view of an embodiment of the invention having multiple facets for sharing single fibers.

FIG. 12B is an illustrative perspective view of the embodiment of FIG. 12A.

FIG. 13A is an illustrative perspective view of an embodiment of the invention for providing groups of multiple facets for sharing single fibers.

FIG. 13B is an illustrative perspective view of the embodiment of FIG. 13A showing a fiber shared between two facets with sample light paths.

FIG. 13C is an illustrative perspective view of an embodiment of the invention having light-blocking separators between groups of multiple facets sharing single fibers.

FIG. 13D is an illustrative perspective view of the embodiment of FIG. 13C showing a fiber shared between two facets with sample light paths.

FIG. 14A is a transverse cross-sectional diagram of sample light-paths according to an embodiment of the invention having multiple facets sharing single fibers.

FIG. 14B, an elevated perspective view of the embodiment of the invention shown in FIG. 14A.

FIG. 15A is an elevated perspective view of the distal portion an endoscope with reflecting elements for delivery and collection of radiation in accordance with the invention.

FIG. 15B is a longitudinal cross-sectional illustrative view of the embodiment shown in FIG. 15A.

FIG. 16 is an elevated perspective view of the distal portion an endoscope integrated with a diffuse light source according to an embodiment of the invention.

### DETAILED DESCRIPTION OF EMBODIMENTS OF THE INVENTION

The accompanying drawings are described below, in which example embodiments in accordance with the present invention are shown. Specific structural and functional details disclosed herein are merely representative. This invention may be embodied in many alternate forms and should not be construed as limited to example embodiments set forth herein.

Accordingly, specific embodiments are shown by way of example in the drawings. It should be understood, however, that there is no intent to limit the invention to the particular forms disclosed, but on the contrary, the invention is to cover all modifications, equivalents, and alternatives falling within the spirit and scope of the claims. Like numbers refer to like elements throughout the description of the figures.

It will be understood that, although the terms first, second, etc. may be used herein to describe various elements, these elements should not be limited by these terms. These terms are used to distinguish one element from another. For example, a first element could be termed a second element, and, similarly, a second element could be termed a first element, without departing from the scope of the present disclosure. As used herein, the term “and/or” includes any and all combinations of one or more of the associated listed items.

It will be understood that when an element is referred to as being “on,” “connected to” or “coupled to” another element, it can be directly on, connected to or coupled to the other element or intervening elements may be present. In contrast, when an element is referred to as being “directly on,” “directly connected to” or “directly coupled to” another element, there are no intervening elements present. Other words used to describe the relationship between elements should be interpreted in a like fashion (e.g., “between” versus “directly between,” “adjacent” versus “directly adjacent,” etc.).

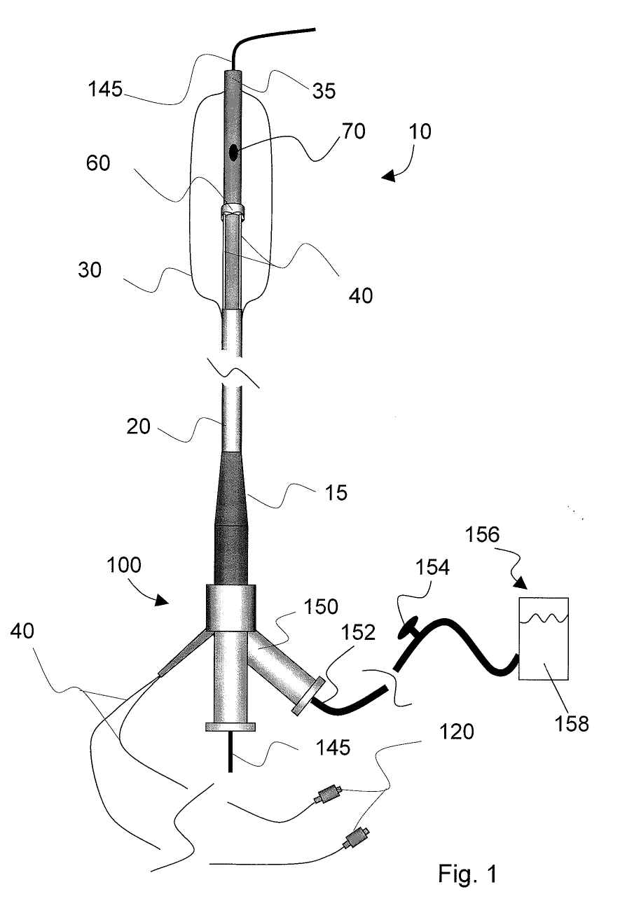

The terminology used herein is for the purpose of describing particular embodiments and is not intended to be limiting of the invention. As used herein, the singular forms “a,” “an” and “the” are intended to include the plural forms as well, unless the context clearly indicates otherwise. It will be further understood that the terms “comprise,” “comprises,” “comprising,” “include,” “includes” and/or “including,” when used herein, specify the presence of stated features, integers, steps, operations, elements, and/or components, but do not preclude the presence or addition of one or more other features, integers, steps, operations, elements, components, and/or groups thereof. FIG. 1 is an illustrative view of a balloon catheter assembly with integrated optics incorporating an embodiment of the present invention.

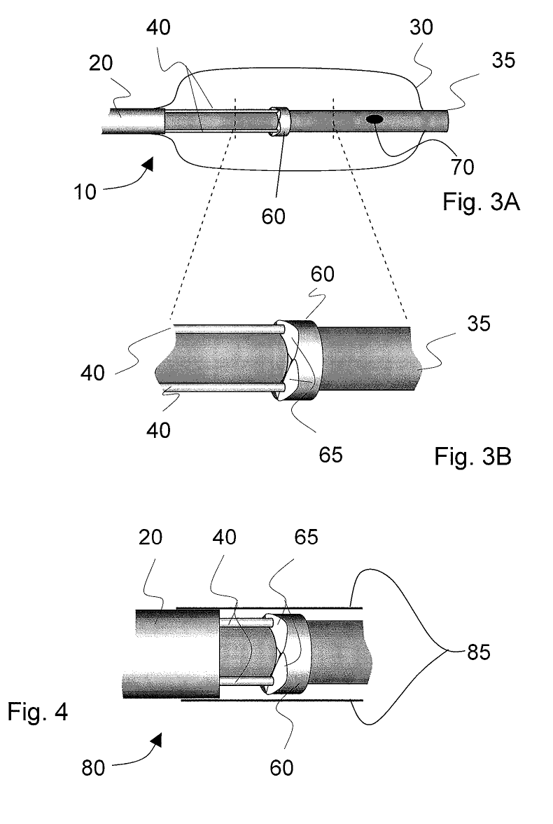

FIG. 1 is an illustrative view of a balloon catheter assembly with integrated optics incorporating an embodiment of the present invention. Referring to FIG. 1, a fiber-optic balloon catheter system 10 is shown incorporating a four-facet embodiment of the invention which is adaptable for inspection and/or treatment of a body lumen wall. The catheter assembly includes a catheter body 20 with fibers 40 and a guidewire sheath 35 with guidewire 145. The distal end of catheter system 10 includes a balloon subassembly (shown in further detail in FIGS. 3A and 3B) with a balloon 30 and a reflecting element 60.

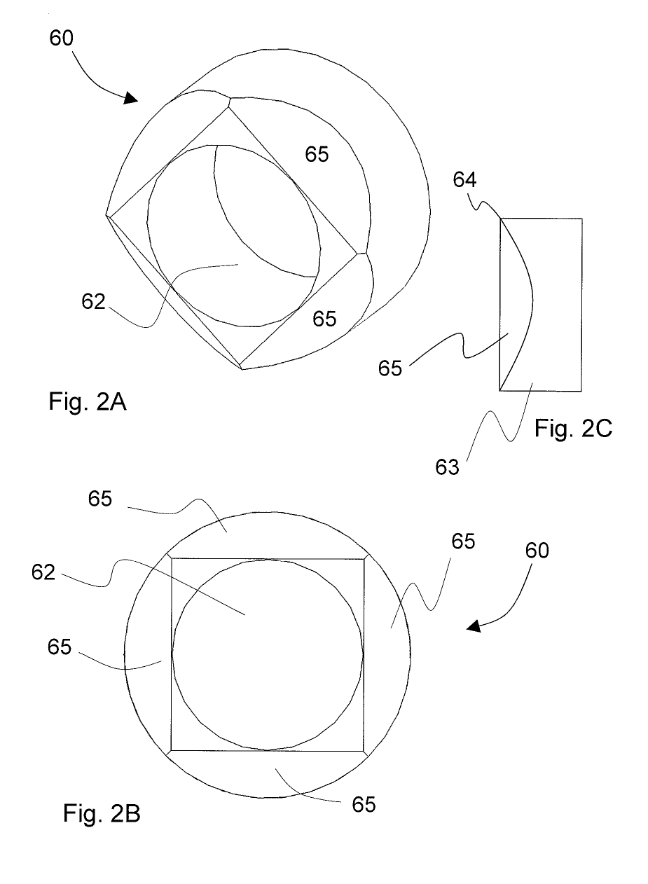

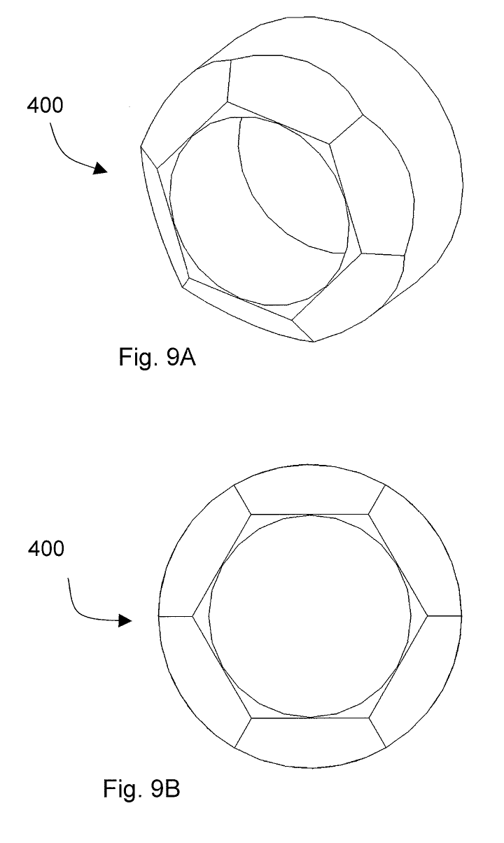

In an embodiment of the invention, the reflecting element 60 can be a four-facet reflecting element, as illustrated in FIGS. 2A-2B. In another embodiment, as shown in FIGS. 9A-9B, the reflecting element 60 can be a six-facet reflecting element.

FIG. 2A is an illustrative elevated perspective view of a reflecting element 60 in accordance with the invention. FIG. 2B is a transverse illustrative view of the reflecting element 60 shown in FIG. 2A. In one example embodiment, the reflecting element 60 is machined, molded, or otherwise formed from a single piece of material, such as metal, polymer, or alloy. In a preferred embodiment, facets 65 are formed out of an end of reflecting element 60. In this manner, facets 65 are unitary with the body of the reflecting element 60. In an embodiment, the end of reflecting element 60 is a longitudinal end or edge 64 of reflecting element 60. In an embodiment, the reflecting element 60 is along a longitudinal axis passing through opening 62 (through which a catheter body can be positioned), and the facets 65 extend outwardly radially from the edge of an inner opening 62 of the element 60. In forming facets 65 from an outer edge 64 of element 60, the facets 65 can be readily formed and shaped according to predetermined light distribution requirements.

In an embodiment, facets 65 are annularly disposed about an opening 62 through which a catheter body passes when assembled and integrated with the catheter (e.g. guidewire sheath 35 as shown in FIG. 1). Facets 65 are oriented at predetermined angles according to a desired predetermined light distribution model. In an embodiment, a surface of each facet 65 extends from an inner surface of the reflecting element 60 to an outer surface of the reflecting element 60. In an embodiment, the inner surface of the reflecting element 60 defines the dimensions of the opening 62, for example, the length and width of the opening 62.

In this manner, when the facets 65 are formed from an end of a standalone, one-piece element 60, and the surface of the facets 65 extends from the inner surface to the outer surface of the reflecting element 60, manufacturing the multi-faceted reflecting element 60 is simplified (e.g. the face of the facets are substantially clear of other components such as alignment grooves). Thus, there is a significantly reduced need for complex, expensive manufacturing techniques and equipment. In addition, this configuration permits better control over alignment of the facets 65 with fibers 40.

In an embodiment, the reflecting element 60 has a maximum outer diameter of about a millimeter or less. In an embodiment, the opening 62 has a diameter of about 0.65 millimeters or less. Facets 65 extend from the inner surface to the outer surface of reflecting element 60, thus allowing for a less costly and complicated manufacturing process and allowing for improved control of the surface properties of facets 65 as further described herein. The relatively small maximum diameter of the embodiments illustrated herein allow for a safe and effective interrogation of relatively small-sized vessels including, for example, coronary and other types of vessels having diameters of 4 millimeters or less and of 2 millimeters or less. Complex, and difficult to manufacture optical components of some other probe catheter designs necessitate larger diameter profiles and thus cannot effectively probe critical vessels having relatively small diameters.

In other embodiments, the reflecting element can include more than four facets, for example, six facets, as illustrated in FIGS. 9A-9B. In other embodiments, the reflecting element can include fewer than six facets, for example, two facets.

In an embodiment, the reflecting element 60 comprises forming a cylindrical ring out of a material, for example, metal. Facets 65 are formed out of the cylindrical ring using, for example, a polisher. The facets 65 can be formed having a flat shape or other shape defined according to one or more predetermined polynomials. In an embodiment, the facets 65 are formed out of a longitudinal end or outermost edge of the cylindrical ring.

Referring again to FIG. 1, the proximate end of balloon catheter system 10 includes a junction 15 attaching catheter body 20 to a connector subassembly 100. Fibers 40 can be fitted with a proprietary or standards-compliant connector 120, for example, face connector/physical contact (FC/PC) type connectors, that are compatible for use with commercially available light sources (not shown) and analyzing devices such as a spectrometer (not shown). Fibers 40 can alternatively be fitted with various commercially distributed multi-port connectors (not shown). Fibers 40 can be delivery fibers (also referred to as source fibers) and/or collection fibers.

Connector subassembly 100 includes a flushing port 150 for delivering a source of liquid/gas 158 to a balloon 30 such as, for example, an angioplasty balloon, through a port 70 in the guidewire sheath 35 in order to expand or contract the balloon 30. In addition, liquid/gas can be removed from the balloon 30 through the port 70 during deflation of the balloon. The source of liquid/gas 158 is held in a tank 156 from which it is pumped into or removed from the balloon 30 through a fluid supply line 152 by actuation of a knob 154. Liquid/gas 158 can alternatively be pumped with the use of automated components (e.g. switches/vacuums). It is preferred that liquid/gaseous solutions for expanding the balloon are non-toxic to humans (e.g. saline solution). It is also preferred that the liquid/gaseous solutions are substantially translucent to the selected light radiation.

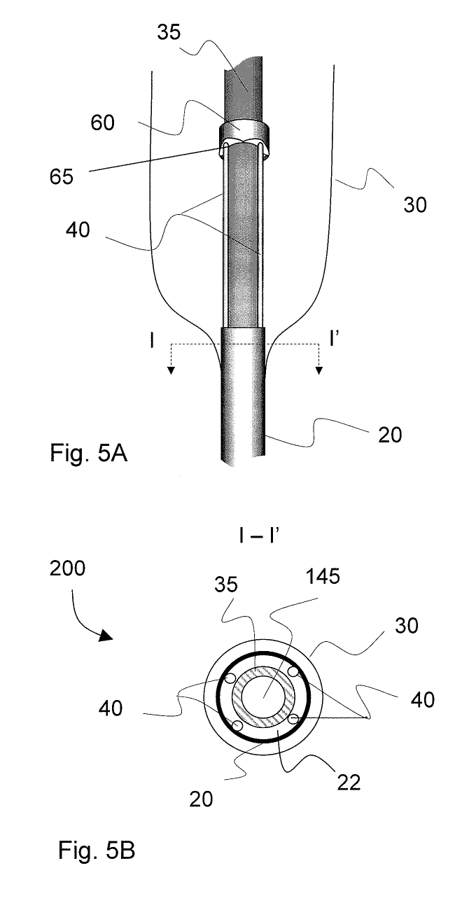

FIG. 3A is an illustrative view of the distal end of a balloon catheter probe with integrated optics in accordance with an embodiment of the invention. FIG. 3B is an expanded illustrative view of the invention as shown in FIG. 3A. Referring to FIGS. 3A-3B, balloon 30 is attached and sealed at its ends to protective catheter body 20 and guidewire sheath 35. A guidewire sheath 35 is formed within catheter body 20 to allow smooth movement of a guidewire 145 within it and then extends out of catheter body 20 into balloon 30. An intervening gap 22 (shown with more clarity in FIG. 5B) is present between catheter body 20 and guidewire sheath 35 through which fibers 40 and liquid/gas 158 pass through. Fibers 40 are secured (e.g. using cement) along guidewire sheath 35 and within catheter body 20 and extend out to reflecting element 60 (shown contained within balloon 30).

In an embodiment, reflecting element 60 includes two or more facets 65, and is secured about guidewire sheath 35. Balloon 30 is preferably made substantially transparent to radiation from sources to fibers 40. Balloon 30 terminates and is attached at its proximate end around catheter body 20 and at its distal end around guidewire sheath 35, physically separating and sealing the fiber optic components from bodily tissues and fluids. Balloon 30 may be attached using well known methods including the use of sealants/adhesives, laser welding, and/or sonic welding.

Fibers 40 include delivery and collection fibers that transmit light exiting and entering the catheter system 10 through balloon 30. Substantially all of the light exiting and entering fibers 40 is reflected from facets 65. The ends of fibers 40, preferably polished to substantially flat smooth surfaces, are affixed to guidewire sheath 35 with the use of cement or other adhesive, and abut, or extend close (e.g. 0.2 mm (200 microns) or less) to the surface of, facets 65.

Radiation sources (not shown) of numerous types (e.g. infrared, near infrared, visible) could be supplied to fibers 40 that are, for example, known to be useful for characterizing various types of tissue conditions or diseased states including those relating to collagen content, lipid content, and calcium content (e.g. via spectral analysis). These radiation types and other types of radiation, including ultraviolet radiation, are also known to be useful for treatment of various tissue conditions which, in an embodiment of the invention, could be delivered through balloon 30. Balloon 30 could alternatively be an expanding angioplasty-type balloon useful for the treatment of blocked, collapsed, or otherwise damaged arteries and useful for anchoring the distal end of the catheter while tissue analysis/treatment is performed with the optics. Port 70 provides a means for supplying a solution (not shown) for expanding and collapsing balloon 30. An analyzer (not shown), such as a spectrometer, is coupled to fibers 40 designated for collecting radiation from tissue. For instance, a spectrometer connected to fibers 40 can be configured to scan radiation across a range of wavelengths that, after interacting with targeted tissue in a body lumen, are known to provide information about the morphology of or presence of various compounds in tissue indicative of healthy or diseased states. For example, spectra within the near infrared spectrum (i.e. 750-5000 nm), and/or visible spectrum (250-750 nm) are known to provide information about lipid content, calcium content, inflammatory factors and/or other factors indicative of stenosis or thrombosis.

FIG. 4 is an illustrative view of the distal end 80 of a catheter probe with integrated optics having a solid transparent window in accordance with an embodiment of the invention. Catheter probe distal end 80, an alternative embodiment to the optics of the catheter shown in FIGS. 2 and 3A-3B, has a solid substantially transparent partial covering 85 instead of the balloon of the prior embodiment. Covering 85 can be manufactured from numerous glasses and plastics, preferably of the type transparent to the radiation selected for analysis. A benefit of this embodiment is that a glass or plastic sheathed assembly may provide more protection for optical components shielded within.

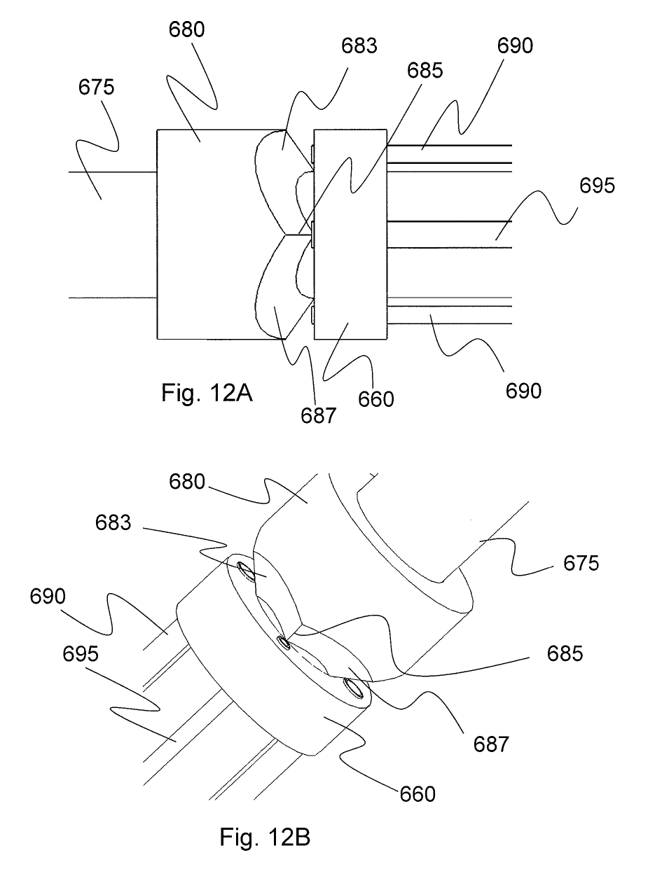

Referring now to FIGS. 5A-5B, the cross section of a packaging arrangement 200 is shown across I-I′ and includes balloon 30 on the outside, within which is catheter sheath 20, within which is guidewire sheath 35, within which is a guidewire 145 that is slideable within guidewire sheath 35. Guidewire 145 can be useful, for example, in positioning the system in accordance with percutaneous transluminal coronary angioplasty (PTCA) by first sliding and guiding guidewire 145 to a location of interest in a coronary artery and then “following” its path with the rest of the catheter. Guidewire sheath 35 secures at least four fiber lines 40 symmetrically distributed about the catheter's central axis, each fiber line 40 corresponding to at least one facet 65 of the reflecting element 60. In an embodiment, as shown in FIGS. 12A-12B, a fiber line can be shared between two or more facets.

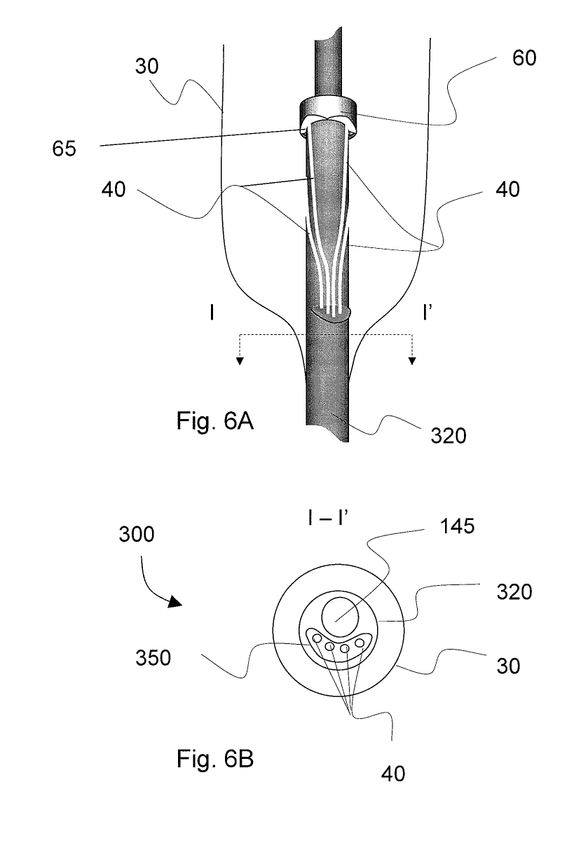

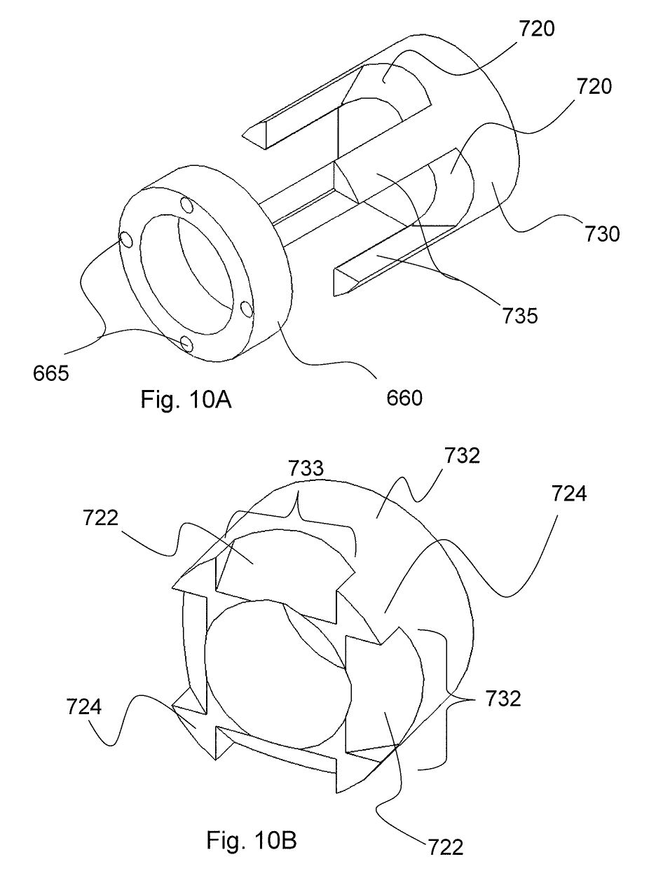

Referring now to FIGS. 6A-6B, an alternative fiber-packaging arrangement 300 is shown across I-I′ wherein fibers 40 are packaged and arranged closely together within an inner fiber sheath 350 contained within guidewire sheath 320. Fibers 40 exit inner sheath 350 within balloon 30 where they are further separated and positioned so that their ends align with corresponding facets 65 on reflecting element 60. In some embodiments, a fiber alignment ring such as, for example, fiber alignment ring 660 as shown in FIGS. 10A, 11A-11C, and 12A-12B, can be used to improve the alignment between fibers 40 and reflecting element 60.

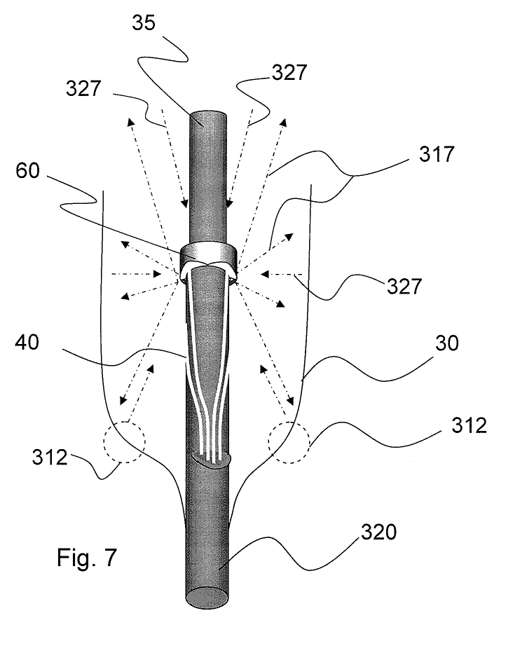

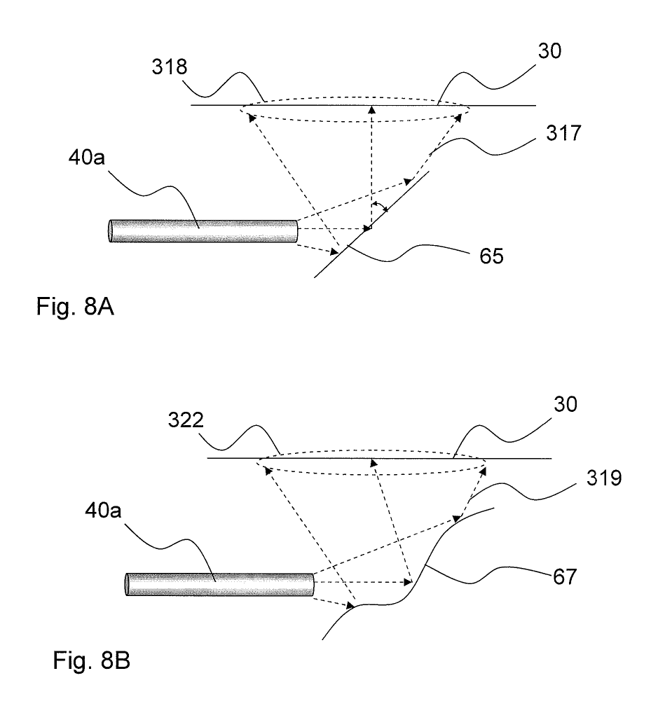

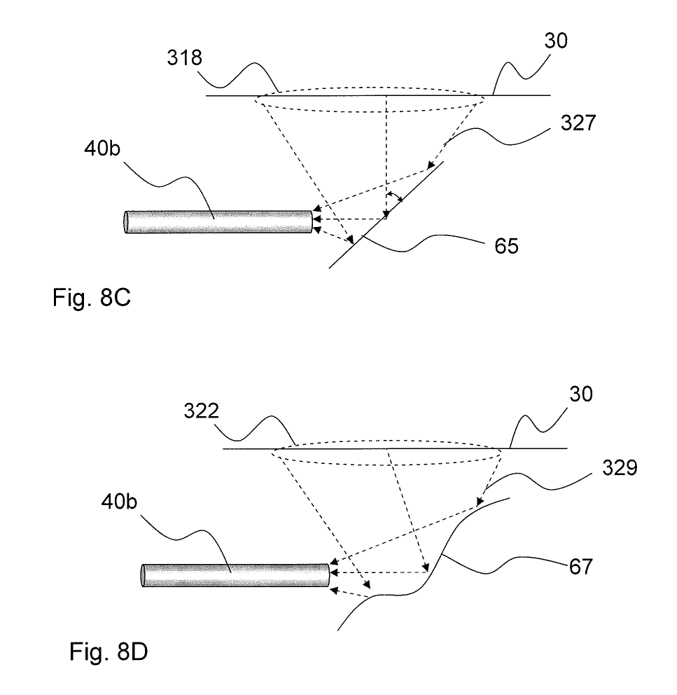

FIG. 7 is an illustrative view of the catheter of FIG. 6A showing sample trace lines 317, 327 of emission and collection rays from fibers 40 in accordance with an embodiment of the invention. FIGS. 8A-8D are illustrative views of sample emission trace lines between one of fibers 40 and a facet 60 according to an embodiment of the invention. More particularly, FIGS. 8A-8B are illustrative views of a delivery fiber 40a transmitting radiation to a target region of a lumen wall, and FIGS. 8C-8D are illustrative views of a collection fiber 40b receiving radiation from a target region of a lumen wall. Referring to FIGS. 7 and 8A-8D, sample emission path ray traces 317, 319 and collection path ray traces 327, 329 are shown from side-view perspectives within balloon 30. Fibers 40a, b along with facets 65, 67 are designed and arranged to project and collect radiation across a predetermined window of the surface of balloon 30. The window preferably consists of those portions that are in contact with the inner surface of a body lumen (not shown). Referring in particular to FIG. 7, the preferred window extends generally between “elbow” portions 312 about the circumference of balloon 30.

In order to achieve the predetermined emission and collection pattern, the numerical aperture (NA) (a factor in the size of the field of views (FOV) 318 and 322) and the positions and angles of fibers 40 (e.g. fibers 40a,b of FIGS. 8A-8D) with respect to facets 65, 67 can be adapted in relation to the size and shape of balloon 30 when expanded. Graded index fibers of high NA, because of their ability to collect greater amounts of light over larger fields, may be used for reducing the number of fibers required in a catheter system and thus reduce the overall diameter of the catheter's distal end. Minimizing the size of the catheter's diameter reduces the stress of passing the catheter through a cardiovascular system and increases its ability to enter through very narrow or partially blocked vessels.

According to embodiments of the invention, each reflecting facet can be planar and angular with respect to a longitudinal axis of a catheter, according to predetermined parameters of the application and desired light distributions. For example, if an application requires the delivery of light at an approximate ninety degree angle from the central axis of the faceted reflecting element, the designated delivery-fiber facets is offset from corresponding delivery fibers at approximately forty five degrees.

In other embodiments of the invention, for example, as illustrated in FIGS. 8B and 8D, the surface of the facets can be shaped according to higher-order polynomials and/or include sub-facets that offer further control over distribution of light emission and collection. If, for example, an application requires a substantially uniform distribution or travel distance between a facet and a target area (e.g., a lumen wall), the shape of the facet could be accordingly designed to accommodate the distribution profile of a fiber and its relationship with the target area. Referring to FIGS. 8B and 8D, a higher-order polynomial shaped facet 67 is shown distributing radiation along predetermined paths 319 to a target area of a lumen wall 330.

FIGS. 9A-9B are illustrative elevated perspective and transverse views, respectively, of a 6-facet reflecting element 400, according to an embodiment of the invention. High numbers of facets may be preferred for systems requiring a greater number of light delivery and collection paths. For example, a system may require numerous discrete areas of interest (to better discern changes among those areas) rather than fewer numbers of broader areas of interest. Lower numbers of facets (e.g., 6 or fewer) may be preferred in embodiments having correspondingly low numbers of fibers so as to minimize the profile and costs of the device within which the faceted element is integrated. Even with low numbers of facets, embodiments of the present invention allow for improved control over the manufacture of the facet surfaces, including their size and shape as further described herein.

In an embodiment, a reflecting element (e.g., such as those described in reference to FIGS. 1-16) is first formed as a ring using any number of well-known techniques including injection molding, forging, and/or lapping, cutting, grinding, and/or polishing. The facets are then formed out of the ring with a polishing instrument appropriate for the material, creating a flat, highly reflecting surface for each facet. The reflecting elements may be formed from any material that offers the required reflecting properties according to the particular application. Materials may include polymers (e.g., plastics) or metals (e.g. stainless steel, gold, titanium, platinum, aluminum, and nickel and/or alloys therefrom). In an embodiment of the invention, the pre-formed ring may first be lightly coated with well known anti-reflecting materials prior to formation of the facets, thus reducing the potential for noise created by light incident on the non-faceted portions.

In an embodiment, the reflecting element and facets such as those described herein may be pre-formed together in a mold and subsequently polished for reflectivity. In another embodiment, the reflecting element is first molded out of plastic of highly reflecting quality and/or subsequently coated with reflecting material (e.g. stainless steel, gold, aluminum, titanium, platinum, nickel and/or alloys therefrom) after which a finishing polish is applied to the facets.

In another embodiment, the reflecting elements are formed as an integrated part of other elements of a device. For example, the guidewire sheath 35 as shown in FIG. 1, and FIGS. 3-7 can be pre-formed together with reflecting element 60 as a single plastic piece. The faceted or non-faceted portions can be similarly coated, polished, and/or shaped as described in earlier embodiments. Certain embodiments of the reflective element may include internal conduits or be internally solid without any openings therein.

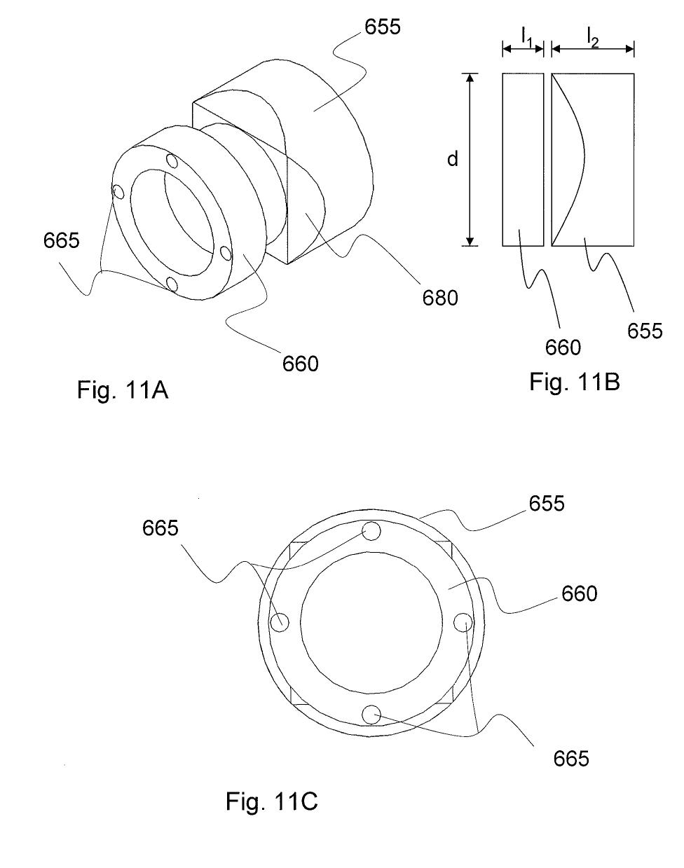

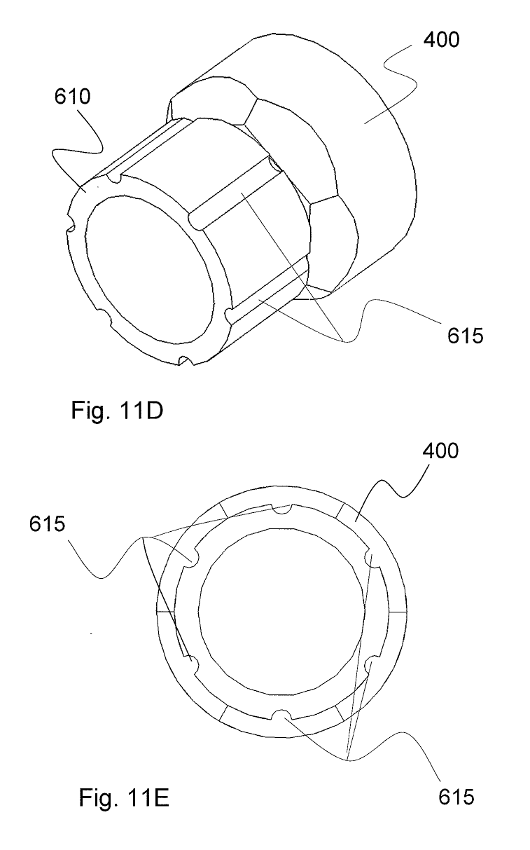

Referring to FIGS. 10A-10B, illustrative elevated perspective views are shown of embodiments of the invention having light-blocking separators positioned between facets. Referring particularly to FIG. 10A, elongated separators 735 help keep radiation which has not first interacted with a targeted area from inadvertently leaking between fibers (not shown) designated for delivery and collection. A fiber alignment segment 660 (described in more detail in reference to FIGS. 11A-11C) can attach and/or be aligned to a faceted segment 730 and align fibers (not shown) passing through alignment holes 665 to facets 720. Separators 735 help keep radiation which has not first interacted with a targeted area from inadvertently leaking between fibers (not shown) designated for delivery and collection. Segments 660 and 730 can be affixed to each other and a catheter body (not shown) passing therethrough. Referring to FIG. 10B, a faceted segment 732 is illustrated in an embodiment of the invention having light-blocking separators 724 and facets 722. Facets 722 have a predetermined width 733 which can be adapted to suit a particular application. Facets 722 can be sized or shaped differently among each other as in previously described embodiments. For example, a facet 722 can be designated for a delivery fiber and have a width (i.e. width 733) smaller or narrower than a width (i.e. width 732) for a facet 722 that is designated for a collection fiber. A larger sized facet, for example, may be appropriate for a collection fiber (as opposed to a delivery fiber) in order to increase the amount of light collected from signals that have generally become weaker than signals transmitted from delivery fibers.

FIG. 11A is an illustrative elevated perspective view of a fiber alignment segment having fiber alignment holes according to an embodiment of the invention. FIG. 11B is a side-perspective view of the embodiment of FIG. 11A. FIG. 11C is an illustrative transverse view of the embodiment of FIG. 11A. The embodiments of FIGS. 11A-11C include a fiber alignment ring 660 with fiber alignment holes 665 for aligning fibers with facets 680 on reflecting element 655. Prior to integration with a catheter, fibers (not shown) can be fed through and positioned within holes 665 of fiber alignment ring 660 and held in place with, for example, an adhesive. The fiber ends can then be polished with a fiber polisher in a manner that is well known to one of ordinary skill in the art. Fiber alignment ring 660 (with fibers in place) can then be assembled onto a catheter system (e.g. placed around a guidewire sheath) to align the fiber's ends with a reflecting element 655 already in place. Cement or another adhesive can be used to hold fiber alignment ring in fixed position relative to reflecting element 665. This feature of positioning, aligning, and then fixing the alignment ring 660 with respect to the reflecting element 655 (see also, FIGS. 11D-11E) provides for a smaller, flexible configuration that is easier to manufacture, and can permit a more accurate alignment between fibers and facets 680 than a one-piece configuration composed of a set of reflectors that extend from fixed alignment grooves within which the locations of fibers cannot be as easily adjusted with respect to facets in an accurate manner.

In an embodiment of the invention, the longitudinal length of the alignment ring 660 (dimension l₁) can be about 350 micrometers or less and the longitudinal length of the reflecting element 665 (dimension l₂) can be about 500 micrometers or less. The diameter of the alignment ring 660 and reflecting element 665 (dimension d) can be about 1 mm or less. An embodiment of the invention can thus provide dynamic optical signal control for a catheter system that is sufficiently flexible and of a small enough profile to traverse narrow blood vessels (e.g. coronary vessels). For example, an embodiment of the invention having a reflective element diameter of about 1 mm can be used to probe or treat vessel sizes of about 2 mm or less.

FIG. 11D-11E are illustrative elevated perspective and transverse views, respectively, of an embodiment of the invention with a distinct alignment segment 610 including fiber retaining grooves 615. Fibers (not shown) can be secured into grooves 615 with an epoxy or other adhesive before or after segment 610 is positioned and aligned with respect to the facets of a multi-faceted segment 400.

FIGS. 12A-12B are illustrated side and elevated perspective views, respectively, of an embodiment of the invention having a fiber shared between multiple facets. A reflecting element 680, a fiber holder 660, and fibers 690 and 695 are integrated with a conduit 675. Fiber 695 can be, for example, a delivery fiber from which radiation can be distributed to both facets 683 and 687 (divided at an intersecting location 685 between the two facets) and subsequently to separate areas of tissue (not shown) near conduit 675. Fibers 690, for example, can be collection fibers which can collect radiation from tissue illuminated by delivery fiber 690. In another embodiment, one or more facets are shared between distribution and collection fibers. Various arrangements for outputting radiation from one or more delivery fibers to one or more facets may provide for more control over distribution, and various arrangements for directing received radiation from one or more facets to one or more collection fibers may provide further control over collection data. In addition, by sharing fibers among multiple facets, the number of fibers incorporated into a catheter or endoscope can be reduced and, thus, can improve the catheter's flexibility and reduce its profile, complexity, and cost.

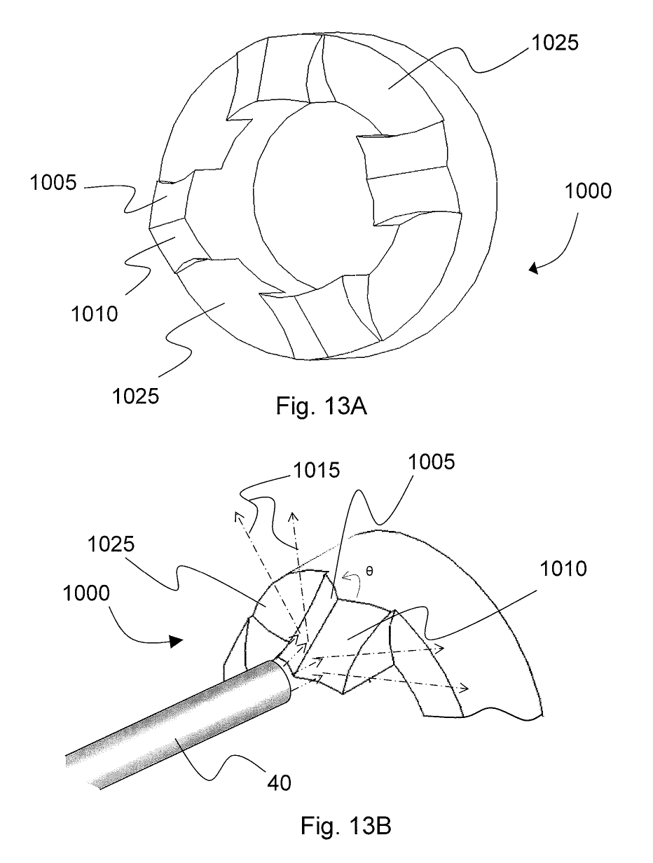

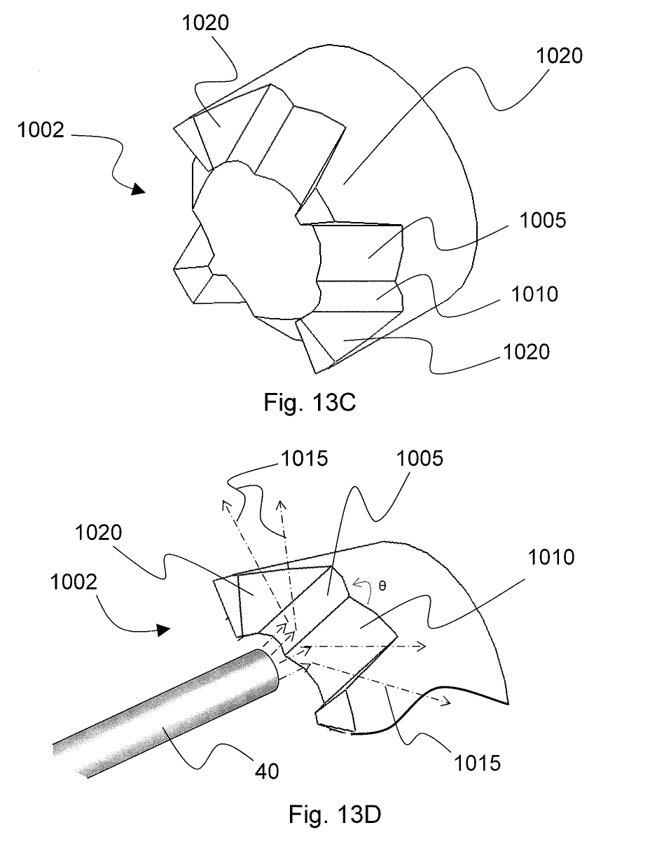

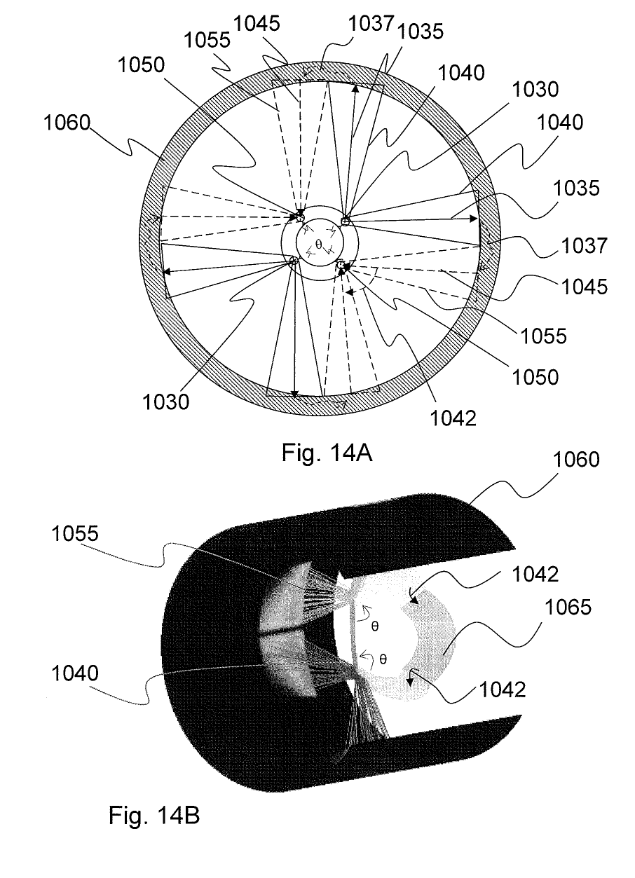

FIGS. 13A-13B are illustrative perspective views of a faceted segment, designated at 1000, in accordance with an embodiment of the invention for providing groups of multiple facets for sharing a single fiber 40. Sample light paths 1015 are shown emanating from fiber 40, then being divided or shared among facets 1005 and 1010 such as, for example, in accordance with sample trace lines shown in FIG. 14. Intervening cavities 1025 are positioned between groups of facets 1005 and 1010. Intervening cavities, such as intervening cavities 1025, can reduce the amount of material used by a faceted segment, thus reducing cost. Providing such cavities can also further simplify the shaping and surfacing of the facets (e.g., facets 1005 and 1010) by minimizing the amount of potentially interfering material about the facet during their shaping and surfacing.

FIGS. 13C-13D are illustrative perspective views of a faceted segment, designated at 1002, in an accordance with an embodiment of the invention having light-blocking separators 1020 between multiple facets 1005 and 1010 designed for sharing a single fiber 40. Sample light paths 1015 are shown emanating from fiber 40, then being divided or shared among facets 1005 and 1010 such as, for example, farther in accordance with sample trace lines shown in FIG. 14. Each of these pairs of facets 1005 and 1010 is separated from other pairs by a separator 1020 to help prevent undesired leaking of radiation between fibers.

FIG. 14A is a transverse cross-sectional view of sample light-paths is shown according to an embodiment of the invention with single fibers shared among multiple (two) facets. FIG. 14B is an elevated perspective view of the embodiment of the invention shown in FIG. 14A. Delivery fibers 1030 direct radiation to two facets of a multi-faceted reflecting element 1065, from which two conical distribution regions 1040 are generated. The facets can be angled and shaped to separate the approximate center of the conical regions by an angle (1042) of, for example, between about 90 and 100 degrees. Thus, in an embodiment of the invention, angle θ can be between about 80 and 90 degrees. A sample ray trace 1035 is shown incident on tissue wall 1060, further distributed within the tissue wall 1060 along a sample path 1037, from which emanates a responsive signal 1045 that becomes incident on multi-faceted reflecting element 1065 and directed to a collection fiber 1050. A conical collection region 1055 is shown from which radiation is collected from tissue wall 1060. In another embodiment of the invention, the angle 1042 between facets can be greater than about 100 degrees such as, for example, up to about 160 degrees and can be as small as about 30 degrees. Increasing the angle 1042 to approximately 120 degrees, for example, can be used to provide increased overlap between conical regions 1040 and 1055 and shorten the path 1037 between distribution and collection of light. Decreasing the angle 1042 can be used to lengthen the path 1037 between distribution and collection of light and increase the depth of signal collected.

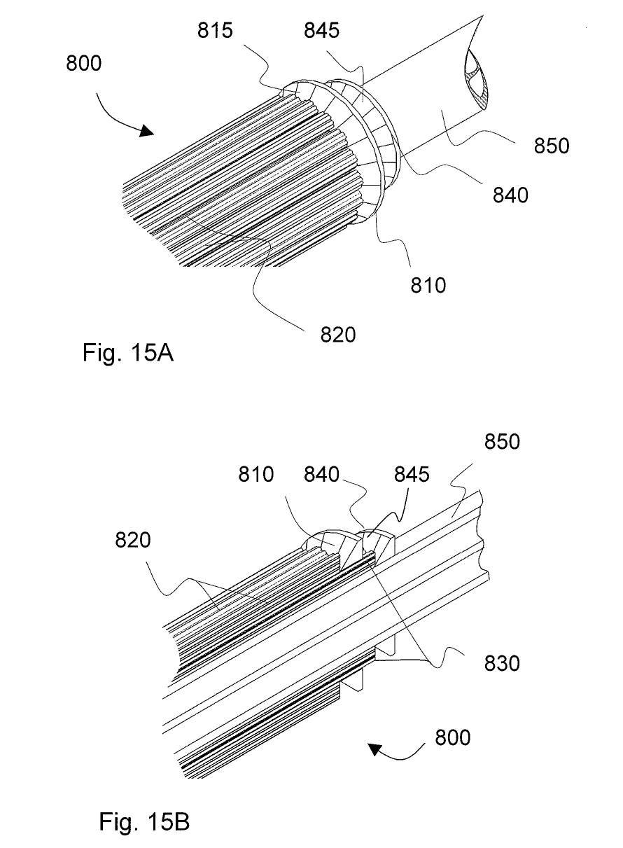

FIG. 15A is an elevated perspective view of the distal portion an endoscope with reflecting elements for delivery and collection of radiation in accordance with embodiments of the invention. FIG. 15B is a longitudinal cross-sectional illustrative view of the embodiment shown in FIG. 15A. Referring to FIGS. 15A-15B, an embodiment of the invention is shown as integrated with the distal portion 800 of an endoscope. The distal portion 800 includes an inner sheath 850 around which is secured a reflecting element 840 and reflecting element 810. Facets 845 of the reflecting element 840 deliver light from delivery fibers 830 to a target image area (not shown) and facets 815 of the reflecting element 810 reflect and transmit images from the target area to collection fibers 820 which collect and direct the images to a viewer and/or imaging device (not shown). Delivery fibers 830 and collection fibers 820 may, respectively, abut facets 845 and 815 or be offset in a predetermined manner from facets 845 and 815. This embodiment permits a simultaneous collection of images to be produced along a 360° arc about the endoscope's distal end (e.g. encompassing an inside cross section of a body lumen), thus reducing a need for mechanical manipulation and/or rotation of the endoscope 800 during inspection. In this manner, images can be collected at the visible light spectrum, i.e., 250 nm.-750 nm.

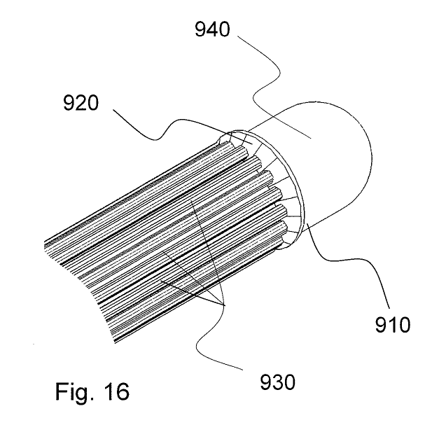

FIG. 16 is an elevated perspective view of the distal portion an endoscope integrated with a diffuse light source in accordance with embodiments of the invention. The distal portion 900 of an endoscope is integrated with a diffuse light source 940 which could include, for example, a lamp enclosed by a light-scattering enclosure (e.g. frosted glass). The diffuse light source 940 outputs radiation in substantially all directions over a large solid angle to a target area, for example, a lumen wall. In an embodiment, the radiation is optical radiation comprising a spectrum of wavelengths, for example, a visible light spectrum ranging from 250-750 nm. Facets 920 reflect and transmit images from the target area to collection fibers 930 which collect and direct the images to a viewer and/or imaging device (not shown).

While the present invention has been particularly shown and described with reference to exemplary embodiments thereof, it will be understood by those of ordinary skill in the art that various changes in form and details may be made therein without departing from the spirit and scope of the present invention as defined by the following claims.

We claim:

1. An endovascular probe assembly for guiding light radiation therein comprising:
at least one waveguide;
a conduit having a longitudinal axis, wherein the at least one waveguide is positioned on a surface of the conduit, and extends along the longitudinal axis of the conduit; and
a reflecting element about the conduit, the reflecting element having a plurality of reflective facets formed out of an end of the reflecting element that are annularly arranged about the conduit, wherein each of the reflective facets is positioned to at least one of direct light to the at least one waveguide and receive reflected light from the at least one waveguide.

2. The probe assembly of claim 1 wherein the reflecting element is substantially cylindrically shaped.

3. The probe assembly of claim 1 wherein the at least one waveguide consists of 4 or fewer waveguides.

4. The probe assembly of claim 3 wherein the 4 or fewer waveguides consists of 4 waveguides.

5. The probe assembly of claim 1 wherein the reflective facets are planar and have predetermined angles with respect to the longitudinal axis of the conduit.

6. The probe assembly of claim 5 wherein at least one of the predetermined angles of one of the reflective facets is distinct from a predetermined angle of at least one other reflective facet of the plurality of reflective facets.

7. The probe assembly of claim 6 wherein the reflective facets are shaped according to one or more predetermined polynomials.

8. The probe assembly of claim 1 wherein the reflecting element is metallic.

9. The probe assembly of claim 1 wherein the reflecting element is comprised of a polymer and the face of each reflective facet includes a highly reflective surface layer.

10. The probe assembly of claim 9 wherein the highly reflective surface layer is selected from the group consisting of steel, nickel, titanium, platinum, aluminum, gold, silver, and alloys therefrom.

11. The probe assembly of claim 1 wherein the reflecting element has a maximum longitudinal length of about half a millimeter or less.

12. The probe assembly of claim 1 wherein the reflecting element includes an opening having an inner surface, wherein the conduit is inserted through the opening, and further includes an outer surface, wherein a surface of each facet extends from the inner surface to the outer surface.

13. The probe assembly of claim 12 wherein the reflecting element has a maximum outer diameter of about a millimeter or less.

14. The probe assembly of claim 1 wherein the reflecting element is contained within a partial covering of the distal end, the partial covering being substantially transparent to a predetermined range of radiation.

15. The probe assembly of claim 14 wherein the partial covering is a flexible angioplasty-type balloon.

16. The probe assembly of claim 1 further comprising an alignment segment for aligning distal ends of the at least one waveguide with the reflective facets.

17. The probe assembly of claim 16 wherein the alignment segment includes one or more grooves for aligning the ends of the at least one waveguide with the reflective facets.

18. The probe assembly of claim 16 wherein the alignment segment includes one or more holes through which the ends of the at least one waveguide can be passed through and aligned with the reflective facets.

19. The probe assembly of claim 16 wherein the alignment segment has a maximum longitudinal length of about 350 micrometers or less.

20. The probe assembly of claim 1 further comprising columns between the reflective facets that substantially block radiation from traveling directly between waveguides corresponding to distinct reflective facets.

21. The probe assembly of claim 1 wherein an intervening cavity is positioned between adjacent reflective facets.

22. The probe assembly of claim 1 wherein at least one individual waveguide is arranged to at least one of deliver light to multiple reflective facets and collect light from multiple reflective facets.

23. The probe assembly of claim 1 wherein at least one facet is of a different size than at least one other facet of the plurality of facets.

24. The probe assembly of claim 23 wherein the at least one facet of a different size is of a larger size than the at least one other facet and is arranged to direct light to a collection fiber.

25. The probe assembly of claim 1 wherein the at least one waveguide is at least one optical fiber.

26. The probe assembly of claim 1 wherein the at least one waveguide and the reflecting element are configured for collecting images.

27. The probe assembly of claim 1 adapted for use in an endoscope.

28. A method of inspecting a body lumen, the method comprising:
providing a source of radiation to a catheter probe having a reflecting element at its distal end, the reflecting element having a plurality of reflective facets that are annularly arranged about the reflecting element and that are formed out of an end of the reflecting element, wherein each reflective facet is shaped to at least one of deliver radiation and receive radiation via one or more corresponding waveguides to or from a target area at predetermined angles of incidence;
inserting the catheter probe into the target area;
collecting through the catheter probe radiation that is received by the reflective facets from the target area;
delivering the received radiation to an analyzer or imager.

29. The method of claim 28 wherein the target area is a body lumen.

30. The method of claim 29 wherein the body lumen has a diameter of about 4 millimeters or less.

31. The method of claim 28 wherein inserting the integrated catheter probe includes:
inserting an angioplasty balloon integrated with the catheter probe, the balloon being substantially transparent to radiation from the catheter's radiation source and wherein the balloon seals the reflecting element from body tissue and fluid; and
prior to receiving the radiation, filling the balloon with non-toxic liquid and expanding the balloon, wherein a substantial portion of the external surface of the balloon is pressed against body tissue.

32. A method for making a fiber-optic probe assembly, the method comprising:
forming a conduit having a longitudinal axis, wherein a waveguide arrangement is positioned on a surface of the conduit, and extends along the longitudinal axis of the conduit;
forming a reflecting element about the conduit, the reflecting element comprising a plurality of reflective facets, wherein the reflective facets are formed out of an end of the reflecting element and arranged annularly about the reflecting element, and wherein each reflective facet is positioned to at least one of direct radiation to the waveguide arrangement and receive light from the waveguide arrangement.

33. The method of claim 32, wherein the waveguide arrangement includes at least one delivery optical fiber and at least one collection optical fiber.

34. The method of claim 32 further comprising forming a protective covering about the reflecting element, the covering being substantially transparent to at least a predetermined range of radiation.

35. The method of claim 32 wherein forming the reflecting element comprises forming a cylindrical ring out of metal and forming flat reflective facets out of an end of the cylindrical ring.

36. The method of claim 32 wherein the flat reflective facets are formed out of the cylindrical ring with a polisher.

37. The method of claim 32 wherein the reflecting element is formed out of a pre-fabricated mold.

38. The method of claim 37 wherein the reflecting element is molded out of plastic and the facets of the plastic, molded reflecting element are subsequently layered with a thin reflecting coating.

39. The method of claim 32 further comprising layering the non-facet surfaces of the reflecting element with an anti-reflecting coating.

40. An endoscope assembly for guiding optical radiation therein comprising:
a conduit along which an waveguide arrangement extends; and
one or more reflecting elements disposed about the conduit, each of the reflecting elements having a plurality of reflective facets annularly arranged about the conduit and configured to at least one of direct light to and receive light from the waveguide arrangement.

41. The endoscope assembly of claim 40 wherein the waveguide arrangement and at least one of the reflecting elements are arranged and configured to collect images.

42. A probe assembly for guiding light radiation therein comprising:
at least one waveguide;
a conduit having a longitudinal axis, wherein the at least one waveguide is positioned on the conduit, and extends along the longitudinal axis of the conduit; and
a reflecting element about the conduit, the reflecting element comprising a opening having an inner surface, wherein the conduit is inserted through the opening, the reflecting element having an outer surface, the reflecting element comprising a plurality of reflective facets, wherein a surface of each facet extends from the inner surface to the outer surface, and wherein each of the reflective facets is positioned to at least one of direct light to the at least one waveguide and receive reflected light from the at least one waveguide.

43. The probe assembly of claim 42 wherein at least one individual waveguide is arranged to at least one of deliver light to multiple reflective facets and collect light from multiple reflective facets.

44. The probe assembly of claim 42 wherein the reflecting element has a maximum longitudinal length of about half a millimeter or less.

45. The probe assembly of claim 42 wherein the reflecting element has a maximum width of about a millimeter or less.

46. The probe assembly of claim 42 wherein the reflecting element comprises a maximum of six facets.

47. The probe assembly of claim 42 wherein the reflecting element comprises a maximum of four facets.
